(19) United States
(12) Patent Application Publication
(10) Pub. No.: US 2006/0159630 A1
(43) Pub. Date: Jul. 20, 2006

(54) Dental material containing bacteristatic and/or bactericidal substances

(76) Inventors: Ingo Haeberlein, Weilheim (DE); Bettina Richter, St. Paul, MN (US)

Correspondence Address: 3M INNOVATIVE PROPERTIES COMPANY, PO BOX 33427, ST. PAUL, MN, 55133-3427, US

(21) Appl. No.: 10/539,952

(22) PCT Filed: Dec. 9, 2003
(86) PCT No.: PCT/EP03/13915

§ 371 Date: Mar. 6, 2006

(30) Foreign Application Priority Data
Dec. 20, 2002 (DE) 102 61 241.2

(51) Int. Cl. A61K 8/49

(52) U.S. Cl. 424/49

(57) ABSTRACT

The invention relates to dental material containing at least one substance, whose bacteriostatic and/or bactericidal activity develops in the presence of intra-oral micro-organisms. The invention also relates to the use of a substance, whose bacterio-static and/or bactericidal activity develops in the presence of intra-oral microorganisms, for producing dental material.

The invention concerns a dental material with bacteriostatic and/or bactericidal substances. The invention further concerns the use of bacteriostatic and/or bactericidal substances for the production of dental materials.

Dental materials with various active ingredients, which feature antimicrobial or bacteriostatic and/or bactericidal properties, are known.

A dental material made of polymer resins is described in EP 0 674 896 B1, which contains a quaternary ammonium compound, which features germicidal and bacteriostatic effects and contains a mixture of essential oils with mint oil, eucalyptus oil, and bergamot oil. The disadvantage of this dental material is the characteristic odor, which can be traced back to the essential oils. Moreover, the use of the quaternary ammonium compound is rejected by experts in part due to the undesirable effect associated with these substances in dental materials and the fact that these materials are intended for lasting placement in the mouth of patients.

Furthermore, the use of the antimicrobial-active substance taurolidine, as well as its metabolite taurultam against periodontosis in the form of a wash solution, is known from DE 26 28 265 C2.

A dental material is disclosed in WO 98/48766, which contains 2,4,4′-trichloro-2′-hydroxydiphenylether (triclosan) as antimicrobial substance. The use of this substance as additive in dental materials causes an antimicrobial effect limited in time since the triclosan is dissolved and moved away as a function of its initial concentration and the saliva flow.

It is therefore the task of this invention to provide a dental material by which the above-mentioned disadvantages of materials known so far are avoided.

This task is solved according to the invention by a dental material containing at least a substance whose bacteriostatic and/or bactericidal effect is developed in the presence of intraoral microorganisms.

The use of these types of substances in dental materials leads to a local and time specific bacteriostatic and/or bactericidal effect from the liberation an active ingredient. This means that this substance initially stores the active ingredient in an inactive form in the dental material. The production, packaging, distribution, as well as storage of the dental material occur with this substance. The active ingredient is therefore present in inactive form. The active ingredient can also be present in this form during the application of the dental material.

The substance contained in the dental material can also additionally feature initial bacteriostatic and/or bactericidal effects, which already develop during the application, for example.

The substance develops its bacteriostatic and/or bactericidal effect by liberating the active ingredient only based on the necessity from external influence, for example, the cultivation of the patient's mouth cavity with pathogenic microorganisms. The same also applies when the dental material comes in contact with microorganisms during the application of the dental material in the patient despite disinfection and sterilization measures, for example, from the dentist's application tools or gloves.

Especially advantageous is in this case that the dental material, which contains the substance liberating the active ingredient, does not affect the patient. The bacteriostatic and/or bactericidal effect of the active ingredient only develops during the formation of the efficacy.

The formation of the efficacy depends on the presence of intraoral microorganisms. It is in particular dependent on the presence of pathogenic and/or undesirable microorganisms in the mouth flora of the patient.

It was determined that, based on the local and time-specific effect of the inventive dental material, its bacteriostatic and/or bactericidal properties only developed at the locations at which the need for bacteriostatic and/or bactericidal effects is given due to the presence of intraoral microorganisms. Thus, the patient was only stressed with active ingredients at those locations and only to the extent that was necessary due to the presence of intraoral microorganisms. This is the case, for example, in a 20 μm wide gap between the dental restoration and the healthy tooth portion. This so-called annular gap can occur between a dental filling, an inlay, an onlay, a crown or bridge, or the dental cement, or bonding consisting of adhesion promoter and the healthy dental substance. For example, polymerization-based shrinkage occurs during the curing of polymer fillings. This annular gap is often populated with intraoral microorganisms after a certain time period after the dental treatment.

Further, the active ingredient only forms in case it is needed. As a result, the concentration of active ingredient is not initially very high and does not diminish with time, but rather the concentration of active ingredient is the highest when the actual need exists due to the presence and growth of intraoral microorganisms. Therefore, the patient is not exposed to a constant, lasting baseline stress from the active ingredient.

The formation of the active ingredient is based on a modification of the substance, for instance, which is caused by enzymatic, physical, chemical, or biochemical changes in the environment. This kind of modification can be a change in the mouth environment from enzyme secretions of the microorganisms.

The environmental change can also result from lysis and the associated liberation of certain substances such as enzymes and metabolites. Furthermore, the enzymes present in the cell wall, the plasma membrane, or in the periplasmatic space of the organism can also cause a chemical change or concentration change in the mouth environment independent of the secretion.

A change in the physical or chemical environmental conditions is also conceivable such as the pH-value, for example, the salt concentration, the temperature, or the like. A modification of the substance is achieved this way by hydrolysis, for example, by transesterification, or changes in the configuration.

A particular advantage of the inventive dental material is that the substance can be enriched and/or stored in the region between the dentin or melt and the dental material. As already described, microorganisms can settle in the annular gap of a filling, for example. The presence of a bacteriostatic and/or bactericidal active ingredient in sufficient concentration is precisely desired in these cases. This can be especially achieved with the inventive dental material due to its enrichment by diffusion of the active ingredient in the areas between the dentine or melt and the dental material. It is ensured in this case with the time and local-specific formation of the active ingredient from the substance that the active ingredient is present in sufficient concentration to achieve a diffusion gradient.

To prevent the diffusion of the substance liberating the active ingredient from the dental material, the substance can be appropriately derivatized. Moreover, it can be covalently bonded in the dental material. These measures provide for the substance to be stored in the area between the dentin or melt and dental material on the surface of the dental material and being locally and time-specifically liberated due to enzymatic, physical, chemical, or biochemical changes in the environment caused from intraoral microorganisms.

In this case, it is possible that the local and time-specific liberation of the substance from the dental material and the development of the efficacy are caused by the same or different type of enzymatic, physical, chemical, or biochemical environmental changes that are triggered by intraoral microorganisms.

It is especially advantageous if the liberation of the substance from the dental material occurs based on enzymatic separation.

Further, the substance can be hindered from diffusing from the dental material due to being derivatized or by being incorporated with covalent bonds into the dental material, and being stored in the area between the dentin or melt and dental material on the surface of the dental material without the substance being liberated. The formation of the efficacy can be based in this case on a modification of the substance, which is caused by enzymatic, physical, chemical, or biochemical environmental changes triggered from intraoral microorganisms, whereby a separation from dental material does not occur.

The formation of the efficacy of a dental material of this kind can occur in this case in multiple steps. For example, they can be the same or different enzymatic, physical, chemical, or biochemical environmental changes triggered by intraoral microorganisms.

Moreover, the substance can also be hindered from diffusion from the dental material after the modification and the associated formation of efficacy by being derivatized, or by being covalently bonded in the dental material.

Substances particularly suited for these purposes comprise, for example taurolidine, and substances, which are present at physiological inactive pH-values of 6-7 and are activated by acidification triggered from metabolic activities of microorganisms, for example the liberation of propionic acid, acetic acid, formic acid, or lactic acid.

Examples of substances/materials that can be utilized are:

Production of taurolidine:

The use of β-azido-ethane-sulfonyl-azide is described in DE 195 15 976 C1 for the production of taurinamide or the production of taurolidine. A method for the production of 2-amino-ethane-sulfonyl-acidic acid addition salts is further described in DE 197 08 872 C1, which then can be converted in known manner to taurolidine or taurultame.

### EXAMPLE 1

### Silicone Based Molding Material

Taurolidine was added to the commercially available Standard-Silicone Molding Material Dimension Penta H (ESPE Dental AG, Seefeld, Germany) whereby taurolidine was added by kneading both to the catalyst paste as well as to the base paste up to a final concentration of 2.5%. Neither the cure behavior nor the storage stability of the silicone molding material was influenced by the taurolidine addition. Circular samples with and without taurolidine addition were produced and each provided with a drop of a culture solution of lactobacillus paracasii. The bactericidal effect of the taurolidine present in the silicone molding material was evaluated under the fluorescence microscope (Axioplan 2, Zeiss Co.) with a commercially available kit from Molecular Probes, Leiden, Netherlands, for life/death determination of bacteria (LIVE/DEAD BacLight Bacterial Viability Kit). The silicone molding material with taurolidine showed about 90% less living bacteria than the silicone molding material without the taurolidine addition.

### EXAMPLE 2

### Molding Material Based on Polyether

Taurolidine was added to the commercially available Standard-Polyether Molding Material Impregum Penta (ESPE Dental Co., Seefeld, Germany) whereby taurolidine was kneaded into both the catalyst as well as the base paste up to a final concentration of 2.5%. The cure behavior was not influenced by the taurolidine addition. Circular samples with and without taurolidine addition were produced and each provided with a drop of a culture solution of lactobacillus paracasii. The bactericidal effect of the taurolidine present in the silicone molding material was evaluated under the fluorescence microscope (Axioplan 2, Zeiss Co.) with a commercially available kit from Molecular Probes, Leiden, Netherlands, for life/death determination of bacteria (LIVE/DEAD BacLight Bacterial Viability Kit). The polyether molding material with taurolidine showed about 90% less living bacteria than the polyether molding material without the taurolidine addition.

### EXAMPLE 3

### Molding Material Based on Alginate

Taurolidine was added to the commercially available Standard-alginate Palgat Quick (ESPE Dental Co., Seefeld, Germany) up to a final taurolidine concentration of 2%. The cure behavior was not influenced by the taurolidine addition. Circular samples with and without taurolidine addition were produced and each provided with a drop of a culture solution of lactobacillus paracasii. The bactericidal effect of the taurolidine present in the alignate material was evaluated under the fluorescence microscope (Axioplan 2, Zeiss Co.) with a commercially available kit from Molecular Probes, Leiden, Netherlands, for life/death determination of bacteria (LIVE/DEAD BacLight Bacterial Viability Kit). The alginate molding material with taurolidine showed living bacteria between 90% to 95% less than the alginate molding material without the taurolidine addition.

### EXAMPLE 4

### Composite-based Filling Material

Taurolidine (<42 μm) was kneaded into the commercially available Composite Filler Material Pertac II (ESPE Dental Co., Seefeld, Germany) up to a final taurolidine concentration of 2.5%. The physical properties of the Pertac II were just slightly influenced by the taurolidine addition. Circular samples with and without taurolidine addition were produced and each provided with a drop of a culture solution of lactobacillus paracasii. The bactericidal effect of the taurolidine present in the glass ionomer cement material was evaluated under the fluorescence microscope (Axioplan 2, Zeiss Co.) with a commercially available kit from Molecular Probes, Leiden, Netherlands, for life/death determination of bacteria (LIVE/DEAD BacLight Bacterial Viability Kit). The composite material with taurolidine showed almost no living bacteria compared to the composite material without the taurolidine addition.

### EXAMPLE 5

### Compomer-based Filling Material

Taurolidine (<42 μm) was incorporated into the commercially available Standard-Compomer Hytac II (ESPE Dental Co., Seefeld, Germany) up to a final taurolidine concentration of 2.5%. The physical properties of the Hytac were only slightly influenced by the taurolidine addition. Circular samples with and without taurolidine addition were produced and each provided with a drop of a culture solution of lactobacillus paracasii. The bactericidal effect of the taurolidine present in the glass ionomer cement material was evaluated under the fluorescence microscope (Axioplan 2, Zeiss Co.) with a commercially available kit from Molecular Probes, Leiden, Netherlands, for life/death determination of bacteria (LIVE/DEAD BacLight Bacterial Viability Kit). The compomer material with taurolidine showed 95% less living bacteria compared to the compomer material without the taurolidine addition.

### EXAMPLE 6

### Filling Material Based on Glass Ionomer Cement

Screened taurolidine (<42 μm) was added to the powdery component of the commercially available Standard Glass Ionomer Vement Ketac Molar (ESPE Dental Co., Seefeld, Germany) up to a final taurolidine concentration of 0.8%. The physical properties of the Ketac were only slightly influenced by the taurolidine addition. Circular samples with and without taurolidine addition were produced and each provided with a drop of a culture solution of lactobacillus paracasii. The bactericidal effect of the taurolidine present in the glass ionomer cement material was evaluated under the fluorescence microscope (Axioplan 2, Zeiss Co.) with a commercially available kit from Molecular Probes, Leiden, Netherlands, for life/death determination of bacteria (LIVE/DEAD BacLight Bacterial Viability Kit). The glass ionomer cement with taurolidine showed approximately 85% less living bacteria than the composite material without the taurolidine addition.

### EXAMPLE 7

### Temporary Filling Materials

Screened taurolidine (<42 μm) was added to the commercially available standard filler material for temporary treatment of cavities Cavit LC (ESPE Dental Co., Seefeld, Germany) up to a final taurolidine concentration of 2.5%. The physical properties of the Cavit LC were only slightly influenced by the taurolidine addition.

Circular samples with and without taurolidine addition were produced and each provided with a drop of a culture solution of lactobacillus paracasii. The bactericidal effect of the taurolidine present in the Cavit LC was evaluated under the fluorescence microscope (Axioplan 2, Zeiss Co.) with a commercially available kit from Molecular Probes, Leiden, Netherlands, for life/death determination of bacteria (LIVE/DEAD BacLight Bacterial Viability Kit). The Cavit LC with taurolidine showed in part absolutely no living bacteria.

### EXAMPLE 8

### Glass Ionomer Mounting Cements

Screened taurolidine (<42 μm) was added to the powdery component of the commercially available standard glass ionomer cement Ketac Cem (ESPE Dental Co., Seefeld, Germany) to a final taurolidine concentration of 2.5% and afterwards mixed until homogeneous. The physical properties of the Ketac Cem were only slightly influenced by the taurolidine addition. Circular samples with and without taurolidine addition were produced and each provided with a drop of a culture solution of lactobacillus paracasii. The bactericidal effect of the taurolidine present in the Cavit was evaluated under the fluorescence microscope (Axioplan 2, Zeiss Co.) with a commercially available kit from Molecular Probes, Leiden, Netherlands, for life/death determination of bacteria (LIVE/DEAD BacLight Bacterial Viability Kit). The glass ionomer mounting cement with taurolidine showed in part absolutely no living bacteria.

### EXAMPLE 9

### Bonding Material

Taurolidine was incorporated into the commercially available standard bonding Visio-Bond (ESPE Dental Co., Seefeld, Germany) up to a final taurolidine concentration of 2.0%. The physical properties of the Visio-Bond were only slightly influenced by the taurolidine addition.

Circular samples with and without taurolidine addition were produced by light exposure of 400 μl Visio-Bond in a 24-size Microtiter plate following the use instructions. The cured platelets were removed and each provided with a drop of a culture solution of lactobacillus paracasii. The bactericidal effect of the taurolidine present in the glass ionomer cement was evaluated under the fluorescence microscope (Axioplan 2, Zeiss Co.) with a commercially available kit from Molecular Probes, Leiden, Netherlands, for life/death determination of bacteria (LIVE/DEAD BacLight Bacterial Viability Kit). The bonding material with taurolidine showed about 90% fewer bacteria compared to the bonding samples without taurolidine addition.

### SUMMARY

The invention concerns a dental material containing at least one substance whose bacteriostatic and/or bactericidal efficacy forms in the presence of intraoral microorganisms.

Further, the invention concerns the use of a substance whose bacteriostatic and/or bactericidal efficacy forms in the presence of intraoral microorganisms for the production of a dental material.

1-16. (canceled)

17. A Dental material comprising at least one substance whose bacteriostatic and/or bactericidal efficacy is formed in the presence of intraoral microorganisms.

18. The Dental material according to claim 1, wherein the formation of the efficacy is based on a modification of the substance, that is caused by an enzymatic, physical, chemical, or biochemical environmental change triggered by the intraoral microorganisms.

19. The Dental material according to one of the claim 1, wherein the substance is enriched and/or stored in the area between the dentin or melt and the dental material.

20. The Dental material according to one of the claim 1, wherein the substance is enriched by diffusion in the area between the dentin or melt and the dental material.

21. The Dental material according to one of the claim 1, wherein the substance is hindered from diffusing from the dental material by being derivatized or being incorporated covalently-bonded in the dental material, and stored in the area between the dentine or melt and the dental material on the surface of the dental material, and by the substance being liberated locally and time-specifically due to enzymatic, physical, chemical, or biochemical environmental changes triggered from intraoral microorganisms.

22. The Dental material according to claim 5, wherein the local and time-specific liberation of the substance and formation of the efficacy can be caused by the same or different enzymatic, physical, chemical, or biochemical environmental changes triggered by intraoral microorganisms.

23. The Dental material according to one of the claim 1, wherein the liberation of the substance occurs based on enzymatic separation.

24. The Dental material according to one of the claim 1, wherein the substance is hindered from diffusion from the dental material by being derivatized or incorporated covalently-bonded in the dental material, and is stored on the surface of the dental material in the area between the dentin or melt and dental material, and formation of the efficacy is based on a modification of the active ingredient which is caused by enzymatic, physical, chemical, or biochemical environmental changes triggered by intraoral microorganisms, whereby the substance is not liberated.

25. The Dental material according to claim 8, wherein the formation of the efficacy occurs in several steps by the same or different enzymatic, physical, chemical, or biochemical environment changes triggered by intraoral microorganisms.

26. The Dental material according to claim 8, wherein the substance remains hindered from diffusing from the dental material after developing the efficacy by being derivatized or incorporated covalently bonded in the dental material.

27. The Dental material according to one of the claim 1, wherein the substance comprises taurolidine.

28. Dental material according to one of the claim 1, comprising
a) 0.01-10% of a substance, whose bacteriostatic and/or bactericidal efficacy is formed in the presence of intraoral microorganisms,
b) 3-80% of a polymerizable component
c) 0.01-25% of typical initiators and/or accelerators and/or retarding agents
d) 0-50% of typical additives
e) 0-90% of typical fillers

29. Dental material according to one of the claim 1, comprising
a) 0.1-5% of a substance, whose bacteriostatic and/or bactericidal efficacy is formed in the presence of intraoral microorganisms,
b) 3-80% of a polymerizable component;
c) 0.01-25% of typical initiators and/or accelerators and/or retarding agents;
d) 0-50% of typical additives; and
e) 0-90% of typical fillers.

30. Dental material according to one of the claim 1, comprising
a) 0.1-3% of a substance, whose bacteriostatic and/or bactericidal efficacy is formed in the presence of intraoral microorganisms;
b) 3-80% of a polymerizable component;
c) 0.01-25% of typical initiators and/or accelerators and/or retarding agents;
d) 0-50% of typical additives; and
e) 0-90% of typical fillers.

31. A method of making a dental material, said method comprising providing a substance whose bacteriostatic and/or bactericidal efficacy forms in the presence of intraoral microorganisms.

32. A method of making a dental molding material, a dental filling material, a glass ionomer cement, a temporary dental filling material, or a dental bonding material, said method comprising providing a substance, whose bacteriostatic and/or bactericidal efficacy forms in the presence of intraoral microorganisms.
